(19) United States
(12) Patent Application Publication
(10) Pub. No.: US 2008/0167476 A1
(43) Pub. Date: Jul. 10, 2008

(54) Process for the preparation of 2-substituted-7-Boryl indoles and compounds

(75) Inventors: Milton R. Smith, East Lansing, MI (US); Robert E. Maleczka, DeWitt, MI (US); Ghayoor Chotana, East Lansing, MI (US); Sulagna Paul, Danbury, CT (US)

Correspondence Address: Ian C. McLeod;IAN C. MCLEOD, P.C., 2190 Commons Parkway, Okemos, MI, 48864, US

(73) Assignee: Board of Trustees of Michigan State University, Lansing, MI (US)

(21) Appl. No.: 11/900,272

(22) Filed: Sep. 11, 2007

Related U.S. Application Data
(60) Provisional application No. 60/843,589, filed on Sep. 11, 2006.

(51) Int. Cl. C07D 209/04

(52) U.S. Cl. 548/405

(57) ABSTRACT

Process for the preparation of 2-substituted-7-boryl indoles and compounds therefrom. The compounds are intermediates to functionalized indoles, both natural and synthetic which are cytotoxic agents, anticancer and antiviral agents.

### CROSS-REFERENCE TO RELATED APPLICATIONS

This application claims benefit to U.S. Provisional Application Ser. No. 60/843,589, filed Sep. 11, 2006, which is incorporated herein by reference in its entirety.

### STATEMENT REGARDING FEDERALLY SPONSORED RESEARCH OR DEVELOPMENT

This work was supported by a grant from the National Institute of Health (NIH)— Grant No. GM063188. The U.S. government has certain rights to this invention.

### BACKGROUND OF THE INVENTION

(1) Field of the Invention

The present invention relates to the preparation of 2-substituted-7-boryl indoles with an unprotected nitrogen, using iridium complexes. The present invention also relates to novel compounds.

(2) Description of the Related Art

Indoles have important biological functions. Traditionally, substituted indole syntheses fall into two classes:¹ (i) Construction of the indole ring system from other substrates,^{1a} and (ii) direct functionalizations of an existing indole.^{1b} The Fischer indole synthesis is an example of the former class, while electrophilic addition is an example of the latter. In this regard, metal-mediated C—H functionalizations of indoles are particularly attractive because elaborations of unprotected indoles are possible.

7-functionalized indoles exist in some intriguing natural products³ such as asperazine,^{3a} chloropeptin I,^{3b} diazonamide A,^{3c} dragmacidin D,^{3d} and TMC-95A and B.^{3e} Because most of these have aryl or alkyl substituents at C7, cross-coupling reactions have been linchpins in synthetic approaches, as indicated in Scheme 1. Since C7 of indole is difficult to functionalize selectively, construction of the requisite coupling partners can be a non-trivial synthetic bottleneck.

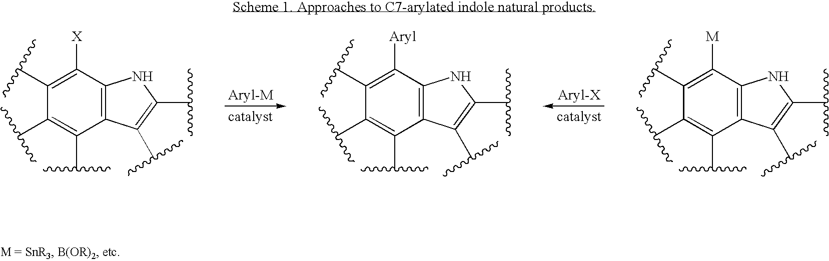

Excluding enzymatic transformations,⁴ indole itself has not been selectively functionalized at C7.⁵ For N-unprotected indoles in general, reactions that functionalize C7 typically generate other isomers or byproducts. The only method with any generality is the N-protection/ortho metallation/electrophilic addition/N-deprotection sequence developed by Snieckus and coworkers.⁶ This method, though, suffers from considerable functional group intolerance. Certainly, similar, but mild, functionalization of unprotected indoles at C7 would have appeal.

U.S. Patent Application No. 2005/0148775 A1 Miyaura et al. describes the preparation of heterocyclic boryl compounds. The preparation of 2-substituted-7-boryl indoles is not described.

### OBJECTS

It is an object of the present invention to provide 2-substituted-7-boryl indoles and a process for the preparation thereof. It is further an object to provide such indoles as intermediates to cytotoxic agents with substituents replacing the boryl groups.

These and other objects will become increasingly apparent by reference to the following description and drawings.

### SUMMARY OF THE INVENTION

The present invention provides a process for producing a 2-substituted-7-boryl indole (I), which comprises: reacting an indole (II) with an unprotected ring nitrogen in a reaction mixture with a non-reactive solvent, selected from, but not limited to, aliphatic hydrocarbons and ethers at temperatures between about 0 and 150° C. with an HB or B-B organic compound, in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium, BY is a boron moiety and the ligand is selected from the group consisting of a phosphorus organic ligand, an organic amine, an imine, a nitrogen heterocycle, and an ether wherein the ligand is at least in part bonded to the iridium, to form the 2-substituted-7-boryl indole (I) in the reaction mixture; and evaporating the solvent and portions of the reaction mixture which are volatile from the reaction mixture to produce the 2-substituted-7-boryl indole (I).

The present invention provides a process for producing a 2-substituted-7-boryl indole (I), which comprises: reacting an indole (II) with an unprotected ring nitrogen with an HB or B-B organic compound in a reaction mixture with a non-reactive first solvent selected from, but not limited to, aliphatic hydrocarbons and ethers at elevated temperatures between about 0 and 150° C. in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium, BY is a boron moiety and the ligand is selected from the group consisting of a phosphorus organic ligand, an organic amine, an imine, a nitrogen heterocycle, and an ether wherein the ligand is at least in part bonded to the iridium, in a molar ratio of complex to ligand between 1 to 3 and 1 to 1, wherein the ligand is at least in part bonded to the iridium, to form the 2-substituted-7-boryl indole (I); evaporating the first solvent and portions of the reaction mixture which are volatile from the reaction mixture; dissolving the 2-substituted-7-boryl indole in a second solvent; and isolating the 2-substituted-7-boryl indole (I) from the second solvent.

The present invention provides a 2-substituted-7-boryl indole (I) with an unprotected ring nitrogen, wherein there is at least one ring substituent in the 2 position (R₁) other than hydrogen selected from the group consisting of boryl, halo, cyano, alkyl, alkoxy, thioalkyl, amino alkyl, acyl, alkyl aminoacyl, trifluoro alkyl, aryl, alkyl silyl, and containing 1 to 8 carbon atoms except for the halo group and boryl group and the boryl group is derived from HBPin or B₂Pin. In further embodiments, the indole (II) is of the formula:

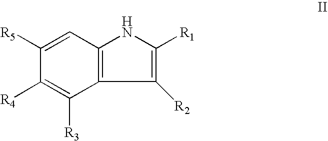

wherein the 2-substituent is R₁ and wherein R₂, R₃, R₄ and R₅ are each selected from the group consisting of hydrogen and the ring substituents for R₁ and wherein the 2-substituted-7-boryl indole (I) is of the formula:

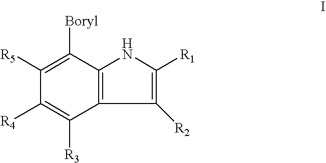

The present invention provides a process for producing 2-substituted-7-boryl indole (I), which comprises: reacting an indole (II) with an unprotected ring nitrogen with HBPin or B₂Pin₂, in a reaction mixture with a non-reactive first solvent selected from, but not limited to, aliphatic hydrocarbons and ethers at elevated temperatures between about 0 and 150° C. in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium BY is a boron moiety and the ligand is selected from the group consisting of a phosphorus organic ligand, an organic amine, an imine, a nitrogen heterocycle, and an ether wherein the ligand is at least in part bonded to the iridium, in a molar ratio of complex to ligand between 1 to 3 and 1 to 1, and wherein the ligand is at least in part bonded to the iridium, to form the 2-substituted-7-boryl indole (I) in the reaction mixture; and evaporating the solvent and portions of the reaction mixture which are volatile from the reaction mixture to produce the 2-substituted-7-boryl indole (I). In further embodiments, there is at least one ring substituent for 2-substituted other than hydrogen selected from the group consisting of boryl, halo other than fluoro, cyano, alkyl, alkoxy, thioalkyl, amino alkyl, acyl, alkyl aminoacyl, trifluoro alkyl, aryl, alkyl silyl, and containing 1 to 8 carbon atoms except for the halo group and boryl group and the boryl group is derived from HBPin or B₂Pin. In further still embodiments, the ligand is selected from the group consisting of:

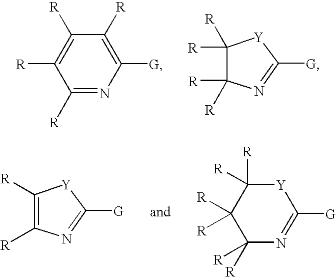

wherein R are each selected from the group consisting of hydrogen, linear alkyl containing 1 to 8 carbon atoms, branched alkyl containing 1 to 8 carbons, and a carbon in a cyclic structure, Y is a carbon, oxygen, nitrogen, or sulfur containing moiety, and G is a heteroatom containing group, multiple atom chain, or multiple atom ring. In further still embodiments, the ligand is selected from the group consisting of:

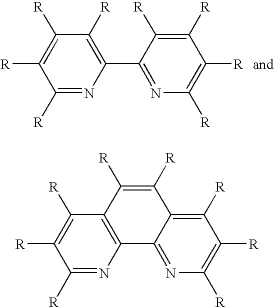

wherein R are each selected from the group consisting of hydrogen, linear alkyl containing 1 to 8 carbon atoms, branched alkyl containing 1 to 8 carbons, and a carbon in a cyclic structure. In still further embodiments, the ligand is selected from the group consisting of:

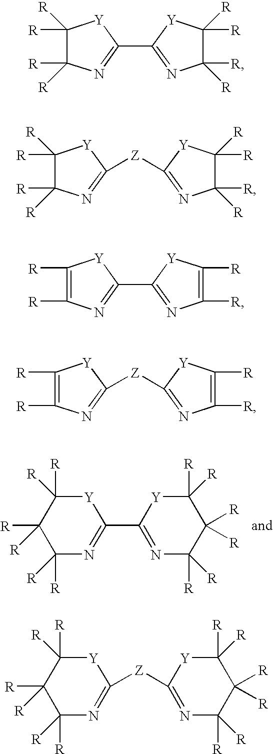

wherein R are each selected from the group consisting of hydrogen, linear alkyl containing 1 to 8 carbon atoms, branched alkyl containing 1 to 8 carbons, and a carbon in a cyclic structure, Y is a carbon, oxygen, nitrogen, or sulfur containing moiety, and Z is a carbon, oxygen, nitrogen, sulfur, or boron containing moiety or a multiple atom chain containing a carbon, oxygen, nitrogen, sulfur, or boron containing moiety. In still further embodiments, the ligand is selected from the group consisting of:

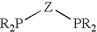

wherein R are each selected from the group consisting of hydrogen, aryl, linear alkyl containing 1 to 8 carbon atoms, branched alkyl containing 1 to 8 carbons, alkoxy, or a carbon in a cyclic structure and Z is a carbon, oxygen, or nitrogen containing moiety or a multiple atom chain containing a carbon, oxygen, or nitrogen containing moiety. In still further embodiments, the HB or B-B organic compound is HBPin or B₂Pin₂. In further still embodiments, the complex is an iridium complex of [Ir(OMe)(COD)]₂, [Ir(Cl)(COD)]₂, or (COD) (η⁵-indenyl)Ir, where COD is 1,5-cyclooctadine, complexed with 4,4-di-t-butyl-2,2′bipyridine (dtbpy). In further embodiments, the complex is an iridium complex of [Ir(OMe)(COD)]₂, [Ir(Cl)(COD)]₂, or (COD)(η⁵-indenyl)Ir, where COD is 1,5-cyclooctadine, complexed with 1,2-bis(dimethylphospino)ethane. In still further embodiments, when indole (I) is reacted with an aryl halide or other suitable cross-coupling electrophile to form a 2-substituted-7-aryl indole.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

Borylations of heterocycles,⁷ small quantities of a single diborylated product arose when indole borylation was carried out with pinacolborane (HBPin) at elevated temperatures. This product could be obtained in good yield by adjusting the HBPin stoichiometry. A series of NMR experiments conclusively identified C7 as the site of the second borylation.

To assess the reaction scope, various substrates and conditions were examined. Bipyridine ligated catalysts disclosed by Ishiyama, Miyaura, and Hartwig performed best.⁸ Of note, moderately elevated temperatures shortened reaction times and improved isolated yields. Because substrates unsubstituted at C2 gave diborylated products (entries 13 and 14) most of the indoles in Table 1 are 2-substituted. Yields were typically good, and the reactions were reasonably functional group tolerant.

Borylations at positions flanking the indole N hint at its participation in the reaction. Three possibilities that we envision are depicted in Scheme 2. In the first pathway, initial N—H scission affords an Ir—N intermediate. Subsequent Ir insertion into the C—H bond at C7 (not shown) would precede product formation. In the second pathway, hydrogen bonding between the hydrogen on the indole nitrogen and a pinacolate oxygen directs C—H insertion. In the third mechanism, coordination of the indole N to Ir directs C—H insertion.

TABLE 1
Ir-catalyzed synthesis of 7-borylated indoles.

| | |
|---|---|
| 1 | 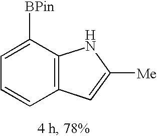 |
| 2 | 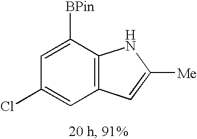 |
| 3 | 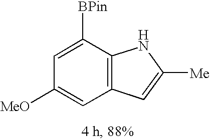 |
| 4 | 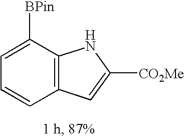 |
| 5^{b} | 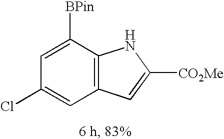 |
| 6^{b} | 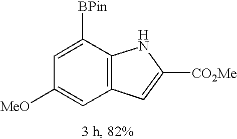 |
| 7^{b} | 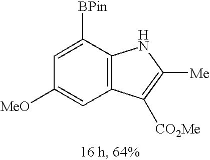 |
| 8^{b} | 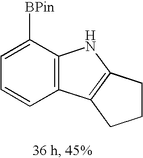 |
| 9 | 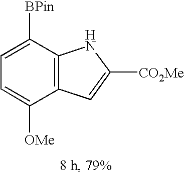 |
| 10^{c} | 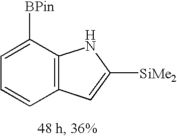 |
| 11 | 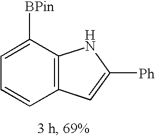 |
| 12^{d} | 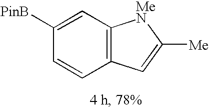 |
| 13^{e} | 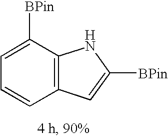 |
| 14^{f} | 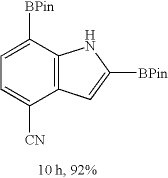 |

In a typical reaction, a solution containing 1.5 equiv HBPin and ≦3 mol % of the pregenerated Ir catalyst was added to the indole.^{8b} The solution was then heated at 60° C. for 3 h. Yields are for isolated products. See Supporting Information for details. ^{c}B₂Pin₂ was the borylating reagent.

To test the first mechanism in Scheme 2, catalytic borylation of N-d₁-5-chloro-2-methylindole and HBPin was examined. At 50% conversion no deuterium incorporation was evident in HBPin or H₂ as judged by ¹H and ¹¹B NMR. This eliminates the specific mechanism in Scheme 2 and argues against other variants predicated on N—H addition.

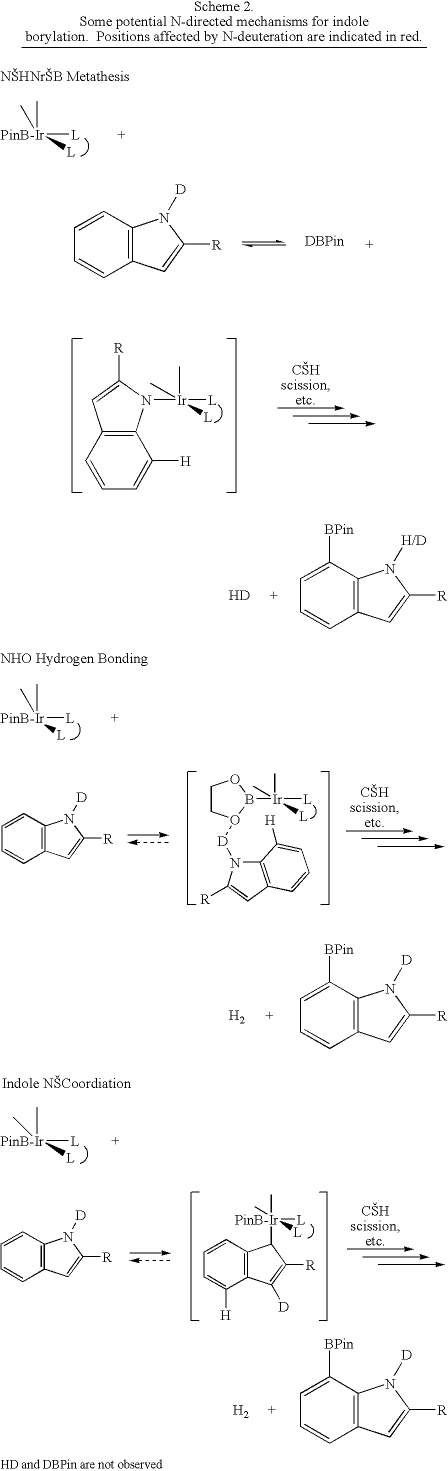

Chart 1. Regiochemistry for the second borylation of benzofuran and indole with the sites of borylation indicated with bold font.

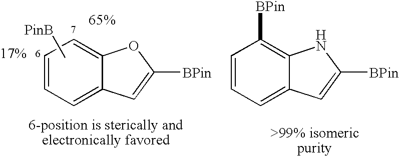

Diborylation of N-methyl indole was initially examined to exclude the second mechanism in Scheme 2. Like indole, initial borylation was observed primarily at C2, but the second borylation occurred mostly at C6. This outcome could be construed as support for H-bonding, but an N-coordination pathway could also be sterically sensitive to methylation.

Because benzofuran is an isosteric analog of indole absent the heteroatom-attached proton, its reactivity can address whether hydrogen bonding is a prerequisite for the indole regioselectivity. As shown in Chart 1, the 2,7- and 2,6-isomers comprise 65% and 17% of the diborylated isomers of benzofuran. Even though the respective meta and para directing effects of OMe and BPin suggest that the second borylation should be favored at C6,¹⁰ the 7-borylated isomer dominates, as was the case for indole.¹¹ Thus, hydrogen bonding to an acidic substrate proton is clearly not required for the observed regioselectivity. Based on these observations, we presently favor the last mechanism in Scheme 2, where N-chelation to Ir (or B) directs borylation.

A recent study raises concerns that the products in Table 1 might perform poorly in Suzuki-Miyaura cross-couplings.¹³ Thus, two one-pot transformations were attempted, where the crude product from Ir-catalyzed borylation was subjected to Pd-catalyzed cross-coupling with an aryl bromide (Scheme 3). Based on the starting indole, the arylated product was isolated in 87% and 82% yield, bolstering the prospects for synthetic utility.

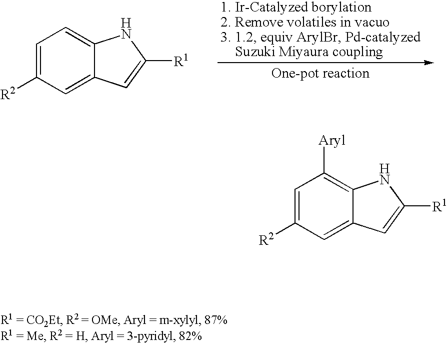

### General Methods:

All commercially available chemicals were used as received unless otherwise indicated. Pinacolborane (HBPin) was generously supplied by BASF. Bis(η⁴-1,5-cyclooctadiene)-di-μ-methoxy-diiridium(I) [Ir(OMe)(COD)]₂ was prepared per the literature procedure.¹ 2-Trimethylsilylindole was prepared per the literature procedure.² Solid substrates were sublimed under vacuum and liquid substrates were purified by distillation. Pinacolborane (HBPin) was distilled before use. n-Hexane was refluxed over sodium, distilled, and degassed. Tetrahydrofuran was obtained from a dry still packed with activated alumina and degassed before use. Silica gel was purchased from EMD™ (230-400 Mesh).

All reactions were monitored by GC-FID (Varian CP-3800; column type: WCOT Fused silica 30 m×0.25 mm ID coating CP-SIL 8 CB). GC-FID method: 70° C., 2 min.; 20° C./min, 9 min.; 250° C., 20 min.; All reported yields are for isolated materials.

¹H and ¹³C NMR spectra were recorded on a Varian Inova-300 (300.11 and 75.47 MHz respectively), Varian VXR-500 or Varian Unity-500-Plus spectrometer (499.74 and 125.67 MHz respectively) and referenced to residual solvent signals (7.24 ppm and 77.0 ppm for CDCl₃, respectively). ¹¹B spectra were recorded on a Varian VXR-300 operating at 96.29 MHz and were referenced to neat BF₃.Et₂O as the external standard. All coupling constants are apparent J values measured at the indicated field strengths. All 2-dimensional experiments were run using z-axis pulse field gradients. Elemental analyses were performed at Michigan State University using a Perkin Elmer Series II 2400 CHNS/O Analyzer. GC-MS data were obtained using a Varian Saturn 2200 GC/MS (column type: WCOT Fused silica 30 m×0.25 mm ID coating CP-SIL 8 CB). High-resolution mass spectra were obtained at the Mass Spectrometry Core of the Research Technology Support Facility (RTSF) at Michigan State University. Melting points were measured on a MEL-TEMP® capillary melting apparatus and are uncorrected.

### General Procedure

Unless otherwise specified, all reactions followed this general procedure. The Ir-catalyst was generated by a modified literature protocol,³ where in a glove box, a Schlenk flask, equipped with a magnetic stirring bar, was charged with the corresponding indole (1 mmol, 1 equiv). Two separate test tubes were charged with [Ir(OMe)(COD)]₂ (10 mg, 0.015 mmol, 3 mol % Ir) and d^{t}bpy (8 mg, 0.03 mmol, 3 mol %). Excess HBPin (1.5 to 2 equiv) was added to the [Ir(OMe)(COD)]₂ test tube. n-Hexane (1 mL) was added to the d^{t}bpy containing test tube in order to dissolve the d^{t}bpy. The d^{t}bpy solution was then mixed with the [Ir(OMe)(COD)]₂ and HBPin mixture. After mixing for one minute, the resulting solution was transferred to the Schlenk flask containing the indole substrate. Additional n-hexane (2×1 mL) was used to wash the test tubes and the washings were transferred to the Schlenk flask. The flask was stoppered, brought out of the glove box, and attached to a Schlenk line in hood. The Schlenk flask was placed under N₂ and heated in a 60° C. oil bath. The reaction was monitored by GC FID/MS. After completion of the reaction, the volatile materials were removed on a rotary evaporator. The crude material was dissolved in CH₂Cl₂ and passed through a short plug of silica to afford the corresponding 7-borylated indole. Small amounts of impurities, if present, were removed by crystallization or column chromatography. Regiochemistry of the borylated products was assigned by NMR spectroscopy (¹H, ¹³C, gHMQC, gHMBC).

### Experimental Details and Spectroscopic Data:

### Table 1, Entry 1: Borylation of 2-methylindole

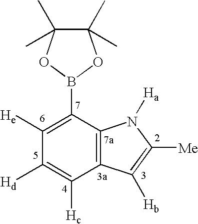

The general procedure was applied to 2-methylindole (262 mg, 2.00 mmol, 1 equiv) and HBPin (435 μL, 384 mg, 3.00 mmol, 1.50 equiv) at 60° C. for 4 h. The product was isolated as colorless oil (201 mg, 78% yield). ¹H NMR (CDCl₃, 300 MHz): δ 8.85 (br s, 1H, Hₐ), 7.62 (d, J=7.8 Hz, 1H, Hₑ), 7.55 (dd, J=7.2, 1.2 Hz, 1H, H_{c}), 7.06 (dd, J=7.8, 7.2 Hz, 1H, H_{d}), 6.21-6.19 (m, 1H, H_{b}), 2.48 (d, J=0.9 Hz, 3H, CH₃), 1.39 (br s, 12H, CH₃ of BPin); ¹³C NMR {H} (CDCl₃, 75 MHz): δ 141.3 (C), 134.9 (C), 128.0 (CH), 127.9 (C), 123.0 (CH), 118.9 (CH), 99.8 (CH), 83.6 (2C), 24.8 (4 CH₃ of BPin), 13.7 (CH₃); ¹¹B NMR (CDCl₃, 96 MHz): δ 31.6; FT-IR (neat) \tilde{v}ₘₐₓ: 3451, 2978, 1604, 1560, 1493, 1431, 1371, 1277, 1136, 974, 850, 802, 752, 679, 636 cm⁻¹; GC-MS (EI) m/z (% relative intensity): M+257 (100), 200 (24); Anal. Calcd for C₁₅H₂OBNO₂: C, 70.06; H, 7.84; N, 5.45. Found: C, 69.68; H, 8.07; N, 5.28.

### Table 1, Entry 2: Borylation of 5-chloro-2-methylindole

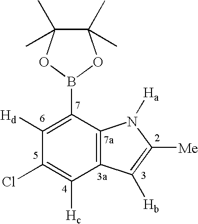

The general procedure was applied to 5-chloro-2-methylindole (166 mg, 1.00 mmol, 1 equiv) and HBPin (218 μL, 192 mg, 1.50 mmol, 1.50 equiv) at room temperature for 20 h. The product was isolated as a white solid (264 mg, 91% yield, mp 106-110° C.). ¹H NMR (CDCl₃, 500 MHz): δ 8.82 (br s, 1H, Hₐ), 7.55 (d, J=1.9 Hz, 1H, H_{c/d}), 7.49 (d, J=1.9 Hz, 1H, H_{c/d}), 6.14 (m, 1H, H_{b}), 2.47 (d, J=0.7 Hz, 3H, CH₃), 1.39 (br s, 12H, CH₃ of BPin); ¹³C NMR {¹H} (CDCl₃, 75 MHz): δ 139.7 (C), 136.7 (C), 129.5 (C), 127.6 (CH), 125.0 (C), 122.3 (CH), 99.6 (CH), 84.2 (2C), 25.0 (4 CH₃ of BPin), 14.0 (CH₃); ¹¹B NMR (CDCl₃, 96 MHz): δ 31.0; FT-IR (neat) \tilde{v}ₘₐₓ: 3443, 2978, 1558, 1464, 1427, 1387, 1130, 952, 881, 850, 758, 679, 640 cm⁻¹; GC-MS (EI) m/z (% relative intensity): M⁺291 (100), 234 (63), 191 (33); Anal. Calcd for C₁₅H₁₉BClNO₂: C, 61.79; H, 6.57; N, 4.80. Found: C, 62.06; H, 6.89; N, 4.78.

### Table 1, Entry 3: Borylation of 5-methoxy-2-methylindole

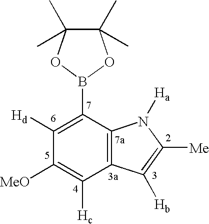

The general procedure was applied to 5-methoxy-2-methylindole (161 mg, 1.00 mmol, 1 equiv) and HBPin (218 μL, 192 mg, 1.50 mmol, 1.50 equiv) at 60° C. for 4 h to give the borylated product (274 mg, 95%) and 3% starting indole. Crystallization from hexanes at −30° C. afforded the desired product as a white solid (253 mg, 88% yield, mp 72-74° C.). ¹H NMR (CDCl₃, 500 MHz): δ 8.72 (br s, 1H, Hₐ), 7.21 (d, J=2.4 Hz, 1H, H_{c/d}), 7.16 (d, J=2.44, 1H, H_{c/d}), 6.14 (m, 1H, H_{b}), 3.86 (s, 3H, OCH₃) 2.47 (d, J=0.7 Hz, 3H, CH₃), 1.39 (br s, 12H, CH₃ of BPin); ¹³C NMR {¹H} (CDCl₃, 75 MHz): δ 153.6 (C), 136.8 (C), 135.9 (C), 128.8 (C), 115.8 (CH), 107.1 (CH), 99.4 (CH), 83.8 (2C), 56.2 (OCH₃), 24.9 (4 CH₃ of BPin), 13.9 (CH₃); ¹¹B NMR (CDCl₃, 96 MHz): δ 31.3; FT-IR (neat) \tilde{v}ₘₐₓ: 3455, 2980, 1483, 1373, 1326, 1282, 1217, 1126, 1041, 852, 752 cm⁻¹; GC-MS (EI) m/z (% relative intensity): M⁺287 (100), 187 (42); Anal. Calcd for C₁₆H₂₂BNO₃: C, 66.92; H, 7.72; N, 4.88. Found: C, 66.68; H, 7.56; N, 4.86.

### Table 1, Entry 4: Borylation of ethyl indole-2-carboxylate

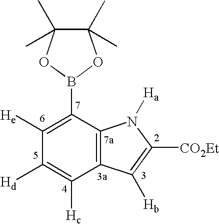

The general procedure was applied to ethyl indole-2-carboxylate (189 mg, 1.00 mmol, 1 equiv) and HBPin (218 μL, 192 mg, 1.50 mmol, 1.50 equiv) at 60° C. for 1 h. The product was isolated as a white solid (274 mg, 87% yield, mp 82-84° C.). ¹H NMR (CDCl₃, 500 MHz): δ 9.69 (br s, 1H, Hₐ), 7.80-7.77 (m, 1H, H_{c}), 7.76 (dd, J=7.0, 1.2 Hz, 1H, Hₑ), 7.20 (d, J=2.2 Hz, 1H, H_{b}), 7.19-7.14 (dd, J=8.1, 7.1 Hz, 1H, H_{d}), 4.40 (q, J=7.2 Hz, 2H, CH₂CH₃), 1.41 (t, J=7.2 Hz, 3H, CH₂CH₃), 1.39 (br s, 12H, CH₃ of BPin); ¹³C NMR {¹H} (CDCl₃, 75 MHz): δ 162.0 (C═O), 141.6 (C), 132.7 (CH), 127.4 (C), 126.4 (C), 126.0 (CH), 120.3 (CH), 108.1 (CH), 84.0 (2C), 60.8 (CH₂), 24.9 (4 CH₃ of BPin), 14.3 (CH₃); ¹¹B NMR (CDCl₃, 96 MHz): δ 31.3; FT-IR (neat) \tilde{v}ₘₐₓ: 3449, 2982, 1711, 1593, 1535, 1421, 1371, 1290, 1232, 1132, 976, 850, 752, 677 cm⁻¹; GC-MS (EI) m/z (% relative intensity): M⁺315 (100), 258 (49), 230 (14), 215 (17), 169(23). Anal. Calcd for C₁₇H₂₂BNO₄; C, 64.78; H, 7.04; N, 4.44. Found: C, 64.45; H, 7.20; N, 4.62.

### Table 1, Entry 5: Borylation of ethyl 5-chloroindole-2-carboxylate

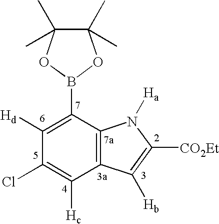

The general procedure was applied to ethyl 5-chloroindole-2-carboxylate (224 mg, 1.00 mmol, 1 equiv) and HBPin (290 μL, 256 mg, 2.00 mmol, 2.00 equiv) at 60° C. for 6 h. The product was isolated as a white solid (290 mg, 83% yield, mp 112-114° C.). ¹H NMR (CDCl₃, 500 MHz): δ 9.64 (s, 1H, Hₐ), 7.73 (dd, J=2.1, 0.7 Hz, 1H, H_{c}) 7.69 (d, J=2.1 Hz, 1H, H_{d}), 7.11 (d, J=2.3 Hz, 1H, H_{b}), 4.40 (q, J=7.1 Hz, 2H, CH₂CH₃), 1.41 (t, J=7.1 Hz, 3H, CH₂CH₃), 1.38 (s, 12H, CH₃ of BPin); ¹³C NMR {¹H} (CDCl₃, 125 MHz): δ 161.6 (C═O), 139.7 (C), 132.5 (CH), 128.6 (C), 127.5 (C), 126.2 (C), 124.8 (CH), 107.3 (CH), 84.4 (2C), 61.0 (CH₂), 24.8 (4 CH₃ of BPin), 14.3 (CH₃); ¹¹B NMR (CDCl₃, 96 MHz): δ 30.9; FT-IR (neat) \tilde{v}ₘₐₓ: 3449, 1709, 1641, 1300, 1233, 1142, 851, 752, 713, 677 cm⁻¹; GC-MS (EI) m/z (% relative intensity): M⁺349 (100), 351 (32), 334 (14), 292 (63); Anal. Calcd for C₁₇H₂₁BClNO₄: C, 58.40; H, 6.05; N, 4.01. Found: C, 58.15; H, 5.79; N, 4.33.

### Table 1, Entry 6: Borylation of ethyl 5-methoxyindole-2-carboxylate

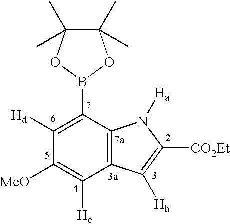

The general procedure was applied to ethyl 5-methoxyindole-2-carboxylate (219 mg, 1.00 mmol, 1 equiv) and HBPin (290 μL, 256 mg, 2.00 mmol, 2.00 equiv) at 60° C. for 3 h. The product was isolated as a white solid (282 mg, 82% yield, mp 79° C.). ¹H NMR (CDCl₃, 500 MHz): δ 9.56 (br s, 1H, Hₐ), 7.44 (d, J=2.4 Hz, 1H, H_{d}), 7.20 (dd, J=2.4, 0.5 Hz, 1H, 1H), 7.11 (d, J=2.3 Hz, 1H, H_{b}), 4.40 (q, J=7.2 Hz, 2H, CH₂CH₃), 3.83 (s, 3H, OCH₃), 1.41 (t, J=7.2 Hz, 3H, CH₂CH₃), 1.38 (br s, 12H, CH₃ of BPin); ¹³C NMR {¹H} (CDCl₃, 125 MHz): δ 162.0 (C═O), 154.3 (C), 137.1 (C), 127.8 (C), 127.1 (C), 122.9 (CH), 107.5 (CH), 107.3 (CH), 84.1 (2C), 60.7 (CH₂), 55.9 (OCH₃), 24.9 (4 CH₃ of BPin), 14.3 (CH₃); ¹¹B NMR (CDCl₃, 96 MHz): δ 31.0; FT-IR (neat) \tilde{v}ₘₐₓ: 3453, 2978, 1705, 1597, 1533, 1446, 1421, 1230, 1213, 1140, 1039, 850, 752 cm⁻¹; GC-MS (EI) m/z (% relative intensity): M⁺ 345 (100), 330 (5), 299 (26), 245 (8), 213 (9), 199 (14); Anal. Calcd for C₁₈H₂₄BNO₅: C, 62.63; H, 7.01; N, 4.06. Found: C, 62.69; H, 7.18; N, 4.20.

### Table 1, Entry 7: Borylation of ethyl 2-methylindole-3-carboxylate

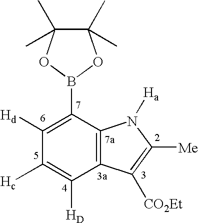

The general procedure was applied to ethyl 2-methylindole-3-carboxylate (203 mg, 1.00 mmol, 1 equiv) and B₂Pin₂ (254 mg, 1.00 mmol, 1.00 equiv, 2.00 equiv of boron) at −60° C. for 18 h. Column chromatography (hexanes/ethyl acetate 90:10) furnished the desired product as a white solid (210 mg, 64% yield, mp 96-98° C.). ¹H NMR (CDCl₃, 500 MHz): δ 9.23 (br s, 1H, Hₐ), 8.19 (m, 1H, H_{c/d}), 7.61 (dd, J=7.1, 1.2 Hz, 1H, H_{b/d}), 7.20 (dd, J=8.0, 7.1 Hz, 1H, H_{c}), 4.38 (q, J=7.2 Hz, 2H, CH₂CH₃), 2.78 (s, 3H, CH₃), 1.43 (t, J=7.2 Hz, 3H, CH₂CH₃), 1.39 (br s, 12H, CH₃ of BPin); ¹³C NMR {¹H} (CDCl₃, 125 MHz): δ 166.1 (C═O), 143.8 (C), 139.8 (C), 129.4 (CH), 126.2 (C), 124.8 (CH), 121.2 (CH), 104.2 (C), 84.0 (2C), 59.3 (CH₂), 25.0 (4 CH₃ of BPin), 14.6 (CH₃), 14.3 (CH₃); ¹¹B NMR CDCl₃, 96 MHz): δ 31.3; FT-IR (neat) \tilde{v}ₘₐₓ: 3429, 2978, 1689, 1591, 1549, 1495, 1373, 1278, 1132, 1093, 846, 804, 756, 680, 652 cm⁻¹; GC-MS (EI) m/z (% relative intensity): M⁺329 (100), 314 (4), 300 (9), 284 (16), 272 (21), 184 (18); Anal. Calcd for C₁₈H₂₄BNO₄: C, 65.67; H, 7.35; N, 4.25. Found: C, 65.47; H, 7.42; N, 4.58.

### Table 1, Entry 8: Borylation of methyl 4-methoxy-2-indolecarboxylate

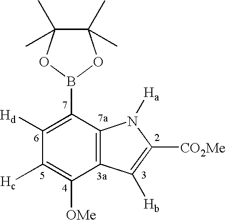

The general procedure was applied to methyl 4-methoxy-2-indolecarboxylate (205 mg, 1.00 mmol, 1 equiv) and HBPin (218 μL, 192 mg, 1.50 mmol, 1.50 equiv) at 60° C. for 8 h, except the starting material was weighed out in test tube and transferred into Schlenk flask by dissolving it into DME (1 mL). The product was isolated as a white solid (261 mg, 79% yield, mp 118-120° C.). ¹H NMR (CDCl₃, 500 MHz): δ 9.66 (br s, 1H, Hₐ), 7.73 (d, J=7.8 Hz, 1H, H_{d}), 7.33 (d, J=2.2 Hz, 1H, H_{b}), 6.51 (d, J=7.8 Hz, 1H, H_{c}), 3.95 (s, 3H, CH₃), 3.92 (s, 3H, CH₃), 1.38 (br s, 12H, CH₃ of BPin); ¹³C NMR {¹H} (CDCl₃, 125 MHz): δ 162.3 (C═O), 157.6 (C), 143.2 (C), 135.0 (CH), 125.6 (C), 117.7 (C), 106.1 (CH), 100.0 (CH), 83.7 (2C), 55.2 (CH₃), 51.7 (CH₃), 24.8 (4 CH₃ of BPin); ¹¹B NMR (CDCl₃, 96 MHz): 831.3; FT-IR (neat) \tilde{v}ₘₐₓ: 3445, 2978, 2841, 1711, 1593, 1531, 1439, 1371, 1182, 1117, 1080, 979, 854, 756, 677 cm⁻¹; GC-MS (EI) m/z (% relative intensity): 1331 (100), 316 (6), 299 (15), 274 (9), 231 (16), 199 (18); Anal. Calcd for C₁₇H₂₂BNO₅: C, 61.65; H, 6.70; N, 4.23. Found: C, 61.58; H, 6.89; N, 4.17.

### Table 1, Entry 9: Borylation of N,N-diethylindole-2-carboxamide

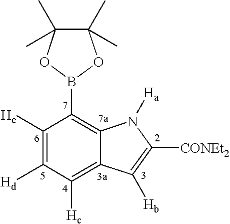

The general procedure was applied to N,N-diethylindole-2-carboxamide (108 mg, 0.5 mmol, 1 equiv) except B₂Pin₂ (127 mg, 0.5 mmol, 2.0 equiv of B) was used instead of HBPin as borylating agent. The reaction was stirred at 60° C. for 2.5 h. Eluting with CH₂Cl₂ through a short silica plug buffered with 2% triethylamine purified the crude and the product was isolated as yellow oil which solidified upon standing (154 mg, 90% yield, mp 108-110° C.). ¹H NMR ((CD₃)₂SO, 500 MHz): δ 9.83 (br s, 1H, Hₐ), 7.80 (d, J=7.9 Hz, 1H, H_{c/e}) , 7.58 (d, J=8.0 Hz, 1H, H_{c/e}), 7.11 (t, J=7.9, 1H, d), 6.89 (d, J=2.2 Hz, 1H, H_{b}), 3.62 (q, J=7.0 Hz, 4H, CH₂CH₃), 1.37 (br s, 12H, CH₃ of BPin), 1.24 (t, J=7.0 Hz, 6H, CH₂CH₃); ¹³C NMR {¹H} ((CD₃)₂SO, 125 MHz): δ 161.0 (C), 139.1 (C), 130.6 (C), 129.3 (CH), 126.5 (CH), 125.1 (C), 119.4 (CH), 103.3 (CH), 83.5 (2C), 41.2 (2 CH₂ of CH₂CH₃), 24.3 (4 CH₃ of BPin), 13.1 (2 CH₃ of CH₂CH₃); ¹¹B NMR (CDCl₃, 96 MHz): δ 31.7; FT-IR (neat) \tilde{v}ₘₐₓ: 3441, 2978, 1616, 1527, 1433, 1369, 1288, 1130, 979, 748, 679 cm⁻¹; GC-MS (EI) m/z (% relative intensity): M⁺342 (100), 270 (67), 243 (79), 186 (22), 170 (54), 142 (12); HRMS (EI): m/z 343.2193 [(M+H)⁺; Calcd for C₁₉H₂₈BN₂O₃: 343.2195].

### Table 1, Entry 10: Borylation of 1,2,3,4-tetrahydrocyclopent[b]indole

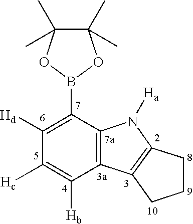

The general procedure was applied to 1,2,3,4-tetrahydrocyclopent[b]indole (157 mg, 1.00 mmol, 1 equiv) and HBPin (290 μL, 256 mg, 2.00 mmol, 2.00 equiv) at 60° C. for 36 h. Column chromatography (hexanes/ethyl acetate 90:10) furnished the desired product as a white solid (127 mg, 45% yield, mp 135° C.). ¹H NMR (CDCl₃, 500 MHz): δ 8.96 (s, 1H, Hₐ), 7.58 (d, J=7.7 Hz, 1H, H_{d}), 7.57 (d, J=7.1 Hz, 1H, H_{b}), 7.1 (dd, J=7.8, 7.2 Hz, 1H, H_{c}), 2.96-2.91 (m, 2H, CH₂), 2.87-2.83 (m, 2H, CH₂), 2.58-2.52 (m, 2H, CH₂), 1.41 (s, 12H, CH₃ of BPin); ¹³C NMR {¹H} (CDCl₃, 125 MHz): δ 146.3 (C), 143.7 (C), 127.5 (CH), 123.6 (C), 121.9 (CH), 119 (C), 118.8 (CH), 83.7 (2C), 28.7 (CH₂), 26.0 (CH₂), 24.9 (4 CH₃ of BPin), 24.4 (CH₂); ¹¹B NMR (CDCl₃, 96 MHz): δ 31.3; FT-IR (neat) \tilde{v}ₘₐₓ: 3453, 3018, 2982, 1414, 1265, 1215, 1136, 929, 848, 767, 669, 623, 509 cm⁻¹; GC-MS (EI) m/z (% relative intensity): M⁺283 (100), 226 (26), 183 (33); HRMS (EI): m/z 283.1743 [(M⁺); calcd for C₁₇H₂₂NO₂B: 283.1744].

### Table 1, Entry 11: Borylation of 2-trimethylsilanylindole

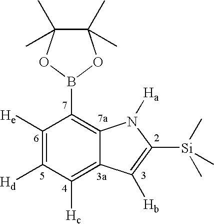

The general procedure was applied to 2-trimethylsilanylindole² (189 mg, 1.00 mmol, 1 equiv) and HBPin (290 μL, 192 mg, 2.00 mmol, 2.00 equiv) at 60° C. for 1 h. The product was isolated as light yellow solid (238 mg, 76% yield, mp 100-102° C.). ¹H NMR (CDCl₃, 300 MHz): δ 9.25 (br s, 1H, Hₐ), 7.79 (d, J=7.8 Hz, 1H, H_{c/e}), 7.67 (d, J=7.1 Hz, 1H, H_{c/e}), 7.13 (dd, J=7.8, 7.1 Hz, 1H, H_{d}), 6.76 (d, J=2.2 Hz, 1H, H_{b}), 1.43 (br s, 12H, CH₃ of BPin), 0.40 (br s, 9H, CH₃ of SiMe₃); ¹³C NMR {¹H} (CDCl₃, 75 MHz): δ 143.7 (C), 137.9 (C), 129.5 (CH), 127.7 (C), 124.1 (CH), 119.2 (CH), 110.5 (CH), 83.7 (2C), 24.9 (4 CH₃ of BPin), −1.1 (3 CH₃ of SiMe₃); ¹¹B NMR (CDCl₃, 96 MHz): δ 31.4; FT-IR (neat) \tilde{v}ₘₐₓ: 3453, 3052, 2978, 1595, 1423, 1368, 1130, 1107, 952, 869, 754, 679 cm⁻¹; GC-MS (EI) m/z (% relative intensity): M⁺315 (100), 301 (27); HRMS (EI): m/z 315.1828 [(M⁺); Calcd for C₁₇H₂₆BNO₂Si: 315.1826].

### Table 1, Entry 12: Borylation of 2-phenylindole

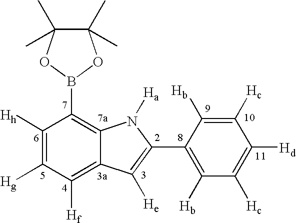

The general procedure was applied to 2-phenylindole (193 mg, 1.00 mmol, 1 equiv) and HBPin (218 μL, 192 mg, 1.50 mmol, 1.5 equiv) at 60° C. for 3 h. The product was isolated as white solid (219 mg, 69% yield, mp 120-121° C.). Small amounts of two isomeric diborylated products were also observed by GC-FID. ¹H NMR (CDCl₃, 500 MHz): δ 9.47 (br s, 1H, Hₐ), 7.76 (d, J=7.8 Hz, 1H, H_{f/h}), 7.71 (d, J=8.4 Hz, 2H, H_{b}), 7.67 (d, J=7.1 Hz, 1H, H_{f/h}), 7.47 (dd, J=8.4, 7.4 Hz, 2H, H_{c}), 7.35 (t, J=7.4 Hz, 1H, H_{d}), 7.15 (dd, J=7.1, 7.8 Hz, 1H, H_{g}), 6.83 (d, J=2.3 Hz, 1H, Hₑ), 1.44 (br s, 12H, CH₃ of BPin); ¹³C NMR {¹H} (CDCl₃, 125 MHz): δ 142.1 (C), 137.6 (C), 132.6 (C), 129.5 (CH), 128.9 (2 CH), 128.2 (C), 127.6 (CH), 125.2 (2 CH), 124.1 (CH), 119.7 (CH), 99.3 (CH), 83.9 (2C), 25.0 (4 CH₃ of BPin); ¹¹B NMR (CDCl₃, 96 MHz): δ 31.7; FT-IR (neat) \tilde{v}ₘₐₓ: 3451, 3055, 2978, 1601, 1498, 1429, 1371, 1323, 1288, 1196, 1145, 1138, 976, 850, 750, 677, 615, 518 cm⁻¹; GC-MS (EI) m/z (% relative intensity): M⁺319 (100), 219 (26); HRMS (EI): m/z 319.1745 [(M⁺); calcd for C₂₀H₂₂BNO₂: 319.1744].

### Table 1, Entry 13: Diborylation of indole

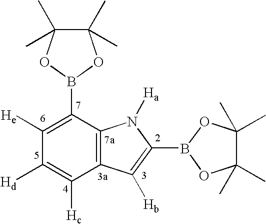

The general procedure was applied to indole (234 mg, 2.00 mmol, 1 equiv) and HBPin (638 μL, 563 mg, 4.40 mmol, 2.20 equiv) using 2 mol % [Ir] catalyst loading at 60° C. for 4 h. After cooling to room temperature, the reaction contents were transferred to a vial. The mother liquor was removed via pipette and the remaining solid was washed with hexanes (2×1 mL). The off-white solid diborylated product (372 mg) was dried under high vacuum. The ¹H NMR spectrum of this solid showed the product to be free of impurities. The washings and the mother liquor were combined, volatile materials were removed under reduced pressure, and the crude mixture was eluted with CH₂Cl₂ through a plug of silica gel to afford additional diborylated product as a white solid (293 mg). Combined yield (665 mg, 90%, mp 147-148° C.). ¹H NMR (CDCl₃, 500 MHz): δ 9.34 (br s, 1H, Hₐ), 7.76 (dt, J=7.9, 1.0 Hz, 1H, H_{c}), 7.68 (dd, J=7.0, 1.2 Hz, 1H, Hₑ), 7.10 (d, J=2.1 Hz, 1H, H_{b}), 7.08 (dd, J=7.9, 7.0 Hz, 1H, 1.40 (br s, 12H, CH₃ of BPin), 1.37 (br s, 12H, CH₃ of BPin); ¹³C NMR {¹H} (CDCl₃, 125 MHz): δ 143.1 (C, C7a), 131.3 (CH, C6), 127.3 (C, C3a), 125.1 (CH, C4), 119.3 (CH, C5), 113.8 (CH, C3), 84.0 (2C), 83.8 (2C), 25.0 (4 CH₃ of BPin), 24.8 (4 CH₃ of BPin); ¹¹B NMR (CDCl₃, 96 MHz): δ 30.1; FT-IR (neat) \tilde{v}ₘₐₓ: 3455, 2978, 1593, 1543, 1371, 1327, 1302, 1262, 1143, 1130, 970, 854, 819, 756, 700, 679 cm⁻¹; GC-MS (EI) m/z (% relative intensity): M⁺369 (100), 312 (4), 285 (7), 269 (4), 254 (2), 226 (2), 212 (4), 184 (3), 169 (3); Anal. Calcd for C₂₀H₂₉B₂NO₄: C, 65.09; H, 7.92; N, 3.80. Found: C, 65.24; H, 8.05; N, 3.65.

¹H, ¹³C NMR, gHMQC and gHMBC spectroscopy (pages S8-S9) showed the single diborylated product to be 2,7-bis(4,4,5,5-tetramethyl-1,3,2-dioxaboryl)-indole as follows. The ¹H NMR spectrum showed that proton H_{d} is ortho coupled to protons H_{c} and Hₑ. This ruled out the possibility of second borylation taking place on C5 or C6. Hence the second borylation took place either at C4 or C7. gHMQC spectrum showed that proton H_{b} (which is coupled to NH proton Hₐ) is attached to C3 at 113.8 ppm. C3 showed a three bond cross peak to proton H_{c} in the gHMBC spectrum. This could only be possible if proton H, is attached to C4. This ruled out the possibility of second borylation taking place at C4. In the ¹³C spectrum of starting indole (and mono borylated indole), carbon C7 is present at 111 ppm. A sharp resonance for C7 was not seen in the ¹³C NMR spectrum of the diborylated product due to broadening from and coupling with boron. Instead quaternary carbon C7 was observed as a broad hump at 111 ppm in the ¹³C spectrum of diborylated product. A three bond cross peak from C7 to proton H_{d} in the gHMBC spectrum was seen as expected for the C7 borylated isomer. Three cross peaks from quaternary carbon C8 to protons H_{b}, H_{c} and Hₑ and one cross peak from quaternary carbon C9 to proton H_{d} in the gHMBC spectrum were also consistent with the second borylation taking place at C7.

Diborylation of indole (using (Ind)Ir(COD) and dmpe at 150° C.).

In a glove box, a Schlenk flask, equipped with a magnetic stirring bar, was charged with indole (234 mg, 2.00 mmol, 1 equiv). (Ind)Ir(COD) (8 mg, 0.02 mmol, 2.00 mol % Ir) and dmpe (3 mg, 0.02 mmol, 2.00 mol %) were weighed in separate test tubes. Excess HBPin (870 μL, 768 mg, 6.00 mmol, 3.00 equiv) was used to dissolve the (Ind)Ir(COD) and dmpe, and the resulting solution was transferred to the Schlenk flask. The flask was stoppered, removed from the glove box, and heated at 150° C. for 1 h. The crude material was dissolved in CH₂Cl₂ and passed through a plug of silica to remove HBPin byproducts. Crystallization from hexane at −80° C. afforded the desired diborylated product as a white solid (516 mg, 70% yield, mp 147-148° C.).

### Table 1, Entry 14: Diborylation of 4-cyanoindole

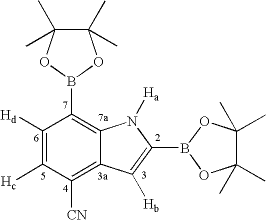

The general procedure was applied to 4-cyanoindole (142 mg, 1.00 mmol, 1 equiv) and HBPin (363 μL, 320 mg, 2.5 mmol, 2.5 equiv) at 60° C. for 10 h. The product was isolated as white solid (362 mg, 92% yield, mp 158-160° C.). ¹H NMR (CDCl₃, 300 MHz): δ 9.50 (br s, 1H, Hₐ), 7.67 (d, J=7.3 Hz, 1H, H_{c/d}), 7.42 (d, J=7.3 Hz, 1H, H_{c/d}), 7.29 (d, J=2.2, 1H, H_{b}), 1.40 (br s, 12H, CH₃ of BPin), 1.37 (br s, 12H, CH₃ of BPin); ¹³C NMR {¹H} (CDCl₃, 125 MHz): δ 142.6 (C), 129.9 (CH), 128.1 (C), 124.4 (CH), 118.4 (C), 112 (CH), 106.4 (C), 85.5 (2C), 84.5 (2C), 24.9 (4 CH₃ of BPin), 24.8 (4 CH₃ of BPin); ¹¹B NMR (CDCl₃, 96 MHz): δ 29.9; FT-IR (neat) \tilde{v}ₘₐₓ: 3445, 2980, 2936, 2218, 1545, 1373, 1332, 1296, 1142, 972, 852, 775, 704, 680 cm⁻¹; GC-MS (EI) m/z (% relative intensity): M⁺394 (100), 379 (8) 337 (16), 309 (10) 294 (7); HRMS (EI): m/z 394.2234 [(M⁺); Calcd for C₂₁H₂₈B₂N₂O₄: 394.2235].

### Desilylation of 7-(4,4,5,5-tetramethyl-1,3,2-dioxaboryl)-2-trimethylsilanylindole

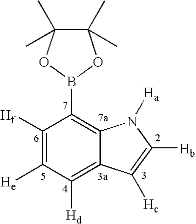

Desilylation of 7-(4,4,5,5-tetramethyl-1,3,2-dioxaboryl)-2 trimethylsilanylindole was carried out as per literature procedure with slight modification.⁴ The silyl compound (157 mg, 0.5 mmol) was dissolved in 1 M tetrabutylammonium fluoride (TBAF) in THF (3 mL). The solution was stirred at 60° C. for 21 h until analysis by GC-FID indicated the absence of starting material. Water (10 mL) was added, and the mixture was extracted with ether (10 mL×3). The combined ether layer was dried over MgSO₄, and the solvent was removed under reduced pressure. The residue was further purified by eluting with CH₂Cl₂ through a plug of silica gel to afford 7-(4,4,5,5-tetramethyl-1,3,2-dioxaboryl-indole as a white solid (107 mg, 88% yield, mp 83-85° C.). ¹H NMR (CDCl₃, 500 MHz): δ 9.24 (br s, 1H, Hₐ), 7.78 (d, J=7.8 Hz, 1H, H_{d/f}), 7.66 (d, J=6.9 Hz, 1H, H_{d/f}), 7.26 (t, J=2.8 Hz, 1H, H_{b}), 7.13 (dd, J=7.8, 6.9 Hz, 1H, Hₑ), 6.55 (m, 1H, H_{c}), 1.39 (br s, 12H, CH₃ of BPin); ¹³C NMR {¹H} (CDCl₃, 125 MHz): δ 140.9 (C), 129.2 (CH), 126.7 (C), 124.2 (CH), 124.0 (CH), 119.3 (CH), 101.9 (CH), 83.8 (2C), 24.9 (4 CH₃ of BPin); ¹¹B NMR (CDCl₃, 96 MHz): δ 31.3; FT-IR (neat) \tilde{v}ₘₐₓ: 3453, 2978, 1635, 1512, 1429, 1369, 1331, 1294, 1130, 974, 798, 729, 679 cm⁻¹; GC-MS (EI) m/z (% relative intensity): M⁺243 (100), 186 (33), 170 (3), 143 (7); HRMS (EI): m/z 243.1431 [(M⁺); Calcd for C₁₄H₁₈BNO₂: 243.1432].

### Monoborylation of Benzofuran

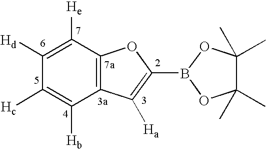

Borylation of benzofuran was carried out using a slightly modified literature procedure.^{3,5} In a glove box [Ir(OMe)(COD)]₂ (10 mg, 0.015 mmol, 3 mol % Ir) and dtbpy (8 mg, 0.03 mmol, 3 mol %) were weighed in separate test tubes. HBPin (175 μL, 154 mg, 1.20 mmol, 1.20 equiv) was added to the [Ir(OMe) (COD)]₂ containing test tube. n-Hexane (1 mL) was added to the dtbpy containing test tube in order to dissolve the dtbpy. The dtbpy solution was then mixed with the [Ir(OMe)(COD)]₂ and HBPin mixture. The resulting solution was transferred to a 20 mL scintillation vial equipped with a magnetic stirring bar. Additional n-hexane (2×1 mL) was used to wash the test tubes and the washings were transferred to the scintillation vial. Benzofuran (108 μL, 118 mg, 1.00 mmol, 1 equiv) was then added to the scintillation vial and the reaction mixture was stirred at room temperature for 30 minutes. GC-FID showed 100% consumption of the starting benzofuran. The ratio of the 2-borylated and 3-borylated benzofuran at the end of reaction was 97.5:2.5 as judged by GC-FID. Volatile materials were removed on a rotary evaporator. The crude material was dissolved in CH₂Cl₂ and passed through a short plug of silica to afford the monoborylated product mixture (199 mg, 82% yield). The minor isomer (2.5% by GC-FID) was not observed by NMR. The following data are for the major (2-borylated) isomer. ¹H NMR (CDCl₃, 300 MHz): δ 7.58-7.62 (m, 1H, H_{b}), 7.55 (dd, J=8.3, 0.7 Hz, 1H, Hₑ), 7.38 (d, J=1.0 Hz, 1H, Hₐ), 7.28-7.34 (m, 1H, H_{d}), 7.20 (dt, J=7.8 Hz, 1.0 Hz, 1H, H_{c}), 1.36 (s, 12H, CH₃ of BPin); ¹³C NMR {¹H} (CDCl₃, 75 MHz): δ 157.5 (C, C7a), 127.5 (C, C3a), 125.9 (CH, C6), 122.7 (CH, C5), 121.8 (CH, C4), 119.5 (CH, C3), 111.9 (CH, C7), 84.6 (2C), 24.7 (4 CH₃ of BPin); ¹¹B NMR (CDCl₃, 96 MHz): δ 28.1; FT-IR (neat) \tilde{v}ₘₐₓ: 3065, 2991, 2978, 2936, 1566, 1361, 1327, 1138, 1068, 962, 852, 831, 819, 756, 748, 692 cm⁻¹; GC-MS (EI) m/z (% relative intensity): M⁺244 (52), 245 (9), 243 (14), 229 (11), 201 (100), 159 (16), 158 (17), 144 (19); Anal. Calcd for C₁₄H₁₇BO₃: C, 68.89; H, 7.02. Found: C, 68.82; H, 7.35.

### Borylation of 2-(4,4,5,5-tetra-ethyl-1,3,2-dioxaboryl)-benzofuran

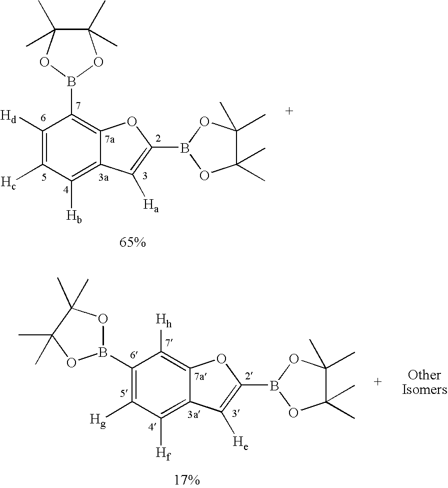

In a glove box 2-borylated benzofuran (containing 2.5% 3-borylated isomer by GC-FID) from the previous reaction (488 mg, 2.00 mmol, 2.00 equiv), [Ir(OMe)(COD)]₂ (10 mg, 0.015 mmol, 3 mol % Ir), and dtbpy (8 mg, 0.03 mmol, 3 mol %) were weighed in separate test tubes. HBPin (145 μL, 128 mg., 1.00 mmol, 1 equiv) was added to the [Ir(OMe)(COD)]₂ containing test tube. n-Hexane (1 mL) was added to the dtbpy containing test tube in order to dissolve the dtbpy. The dtbpy solution was then mixed with [Ir(OMe)(COD)]₂ and HBPin mixture. The resulting solution was transferred to a 20 mL scintillation vial equipped with a magnetic stirring bar. To this vial was added the n-hexane solution (1 mL) of the monoborylated benzofuran. Additional n-hexane (1 mL) was used to wash the test tubes and the washings were transferred to the scintillation vial. The reaction mixture was stirred at room temperature. The reaction was stopped after 7 hours to minimize any conversion of diborylated products in to triborylated products. Six diborylated products were observed by GC-FID, their GC retention times and percentage of the isomer mixture are given in the following table.

| Isomer | A | B | C | D | E | F |
|---|---|---|---|---|---|---|
| GC ret. Time | 14.54 | 14.81 | 15.71 | 15.92 | 17.06 | 17.58 |
| (min) | | | | | | |
| GC | 64.5 | 3.4 | 4.2 | 7.5 | 3.9 | 16.6 |
| Percentage | | | | | | |

After the reaction was stopped, the volatile materials were removed on a rotary evaporator. Gradient column chromatography (hexanes:CH₂Cl₂ 50:50-→hexanes:CH₂Cl₂ 0:100) was used to isolate the major diborylated product (136 mg, 37%, GC-FID retention time 14.54 min). This fraction was of ≅92% isomeric purity by GC-FID. Crystallization from n-hexanes at −80° C. afforded the pure diborylated product as a white solid (90 mg, 24% yield, mp 161-162° C.). ¹H, ¹³C NMR, gHMQC and gHMBC spectroscopy showed this major diborylated isomer to be 2,7-bis(4,4,5,5-tetramethyl-1,3,2-dioxaboryl)-benzofuran. ¹H NMR (CDCl₃, 500 MHz): δ 7.77 (dd, J=7.2, 1.3 Hz, 1H, H_{d}), 7.70 (dd, J=7.8, 1.3 Hz, 1H, H_{b}), 7.36 (s, 1H, Hₐ), 7.21 (dd, J=7.8, 7.2 Hz, 1H, Hₐ), 1.40 (s, 12H, CH₃ of BPin), 1.35 (s, 12H, CH₃ of BPin); ¹³C NMR {¹H} (CDCl₃, 125 MHz): δ 161.5 (C, C7a), 133.4 (CH, C6), 127.0 (C, C3a), 125.1 (CH, C4), 122.2 (CH, C5), 119.5 (CH, C3), 84.4 (2C), 84.0 (2C), 24.9 (4 CH₃ of BPin), 24.8 (4 CH₃ of BPin); ¹¹B NMR (CDCl₃, 96 MHz): δ 30.6; FT-IR (neat) \tilde{v}ₘₐₓ: 3063, 2980, 2934, 1608, 1591, 1570, 1487, 1363, 1338, 1307, 1165, 1143, 1130, 1072, 980, 922, 850, 760, 694, 679 cm⁻¹; GC-MS (EI) m/z (% relative intensity): M⁺370 (100), 371 (20), 369 (48), 355 (5), 284 (20), 270 (8), 227 (40); Anal. Calcd for C₂₀H₂₈B₂O₅: C, 64.91; H, 7.63. Found: C, 64.64; H, 7.98.

Another fraction from the column was obtained (21.0 mg) which was significantly enriched in isomer F (≅66% by GC-FID retention time 17.58 min). This sample was sufficiently enriched in isomer F to identify and assign its regiochemistry by NMR. ¹H, ¹³C NMR, gHMQC and gHMBC spectroscopy showed this minor diborylated isomer F (GC-FID retention time 17.58 min) to be 2,6-bis(4,4,5,5-tetramethyl-1,3,2-dioxaboryl)-benzofuran. ¹H NMR (CDCl₃, 500 MHz): δ 7.96 (d, J=0.9 Hz, 1H, H_{b}), 7.65 (dd, J=7.8, 0.7 Hz, 1H_{g}), 7.60 (dd, J=7.8, 0.7 Hz, 1H, H_{f}), 7.37 (d, J=1.1 Hz 1H, Hₑ), 1.37 (s, 12H, CH₃ of BPin), 1.34 (s, 12H, CH₃ of BPin); ¹³C NMR {¹H} (CDCl₃, 125 MHz): δ 157.4 (C, C7a′), 130.1 (C, C3a′), 128.7 (CH, C5′), 121.2 (CH, C4′), 119.6 (CH, C3′), 118.1 (CH, C7′), 84.7 (2C), 83.8 (2C), 24.9 (4 CH₃ of BPin), 24.8 (4 CH₃ of BPin).

Unreacted 2-(4,4,5,5-tetramethyl-1,3,2-dioxaboryl)-benzofuran was also recovered (277 mg, 57%).

### One-Pot Borylation/Suzuki Coupling of ethyl 5-methoxyindole-2-carboxylate

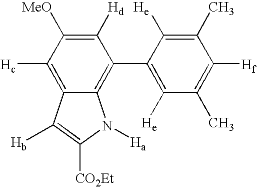

The general borylation procedure was applied to ethyl 5-methoxyindole-2-carboxylate (219 mg, 1.00 mmol, 1 equiv) and HBPin (290 μL, 256 mg, 2.00 mmol, 2.00 equiv) at 60° C. for 3 h. The GC-FID showed 100% consumption of the starting indole. The reaction mixture was pumped down under high vacuum for 1 h to remove the volatile materials. The Schlenk flask was brought into the glove box, where Pd(PPh₃)₄ (12 mg, 1 mol %), 5-bromo-m-xylene (163 μL, 222 mg, 1.20 equiv), and DME (3 mL) were added. The Schlenk flask was then brought out of the glove box and attached to a Schlenk line. K₃PO₄.nH₂O (319 mg, 1.50 equiv) was added under N₂ counter flow to the Schlenk flask. The flask was stoppered and the mixture was heated at 80° C. for 2 h. The flask was cooled down to room temperature and 5 mL of water were added to the reaction mixture. The reaction mixture was extracted with ether (10 mL×3). The combined ether extractions were washed with brine (10 mL), followed by water (10 mL), dried over MgSO₄ before being concentrated under reduced pressure on a rotary evaporator. Column chromatography (CH₂Cl₂) furnished the desired product as a white solid (280 mg, 87% yield, R_{f}=0.6, mp 125-126° C.). ¹H NMR (CDCl₃, 300 MHz): δ 8.89 (br s, 1H, Hₐ), 7.22-7.21 (m, 2H, Hₐ), 7.18 (d, J=2.2 Hz, 1H, H_{c/d}), 7.07-7.06 (m, 1H, H_{f}), 7.04 (d, J=2.2 Hz, 1H, H_{c/d}), 7.00 (d, J=2.4 Hz, 1H, H_{b}), 4.40 (q, J=7.1 Hz, 2H, CH₂CH₃), 3.87 (s, 3H, OCH₃), 2.40 (s, 6H, CH₃ of xylene), 1.40 (t, J=7.1 Hz, 3H, CH₂CH₃); ¹³C NMR {¹H} (CDCl₃, 75 MHz): δ 161.9 (C), 155.1 (C), 138.9 (2C), 138.0 (C), 130.6 (C), 129.7 (CH), 128.3 (C), 128.1 (C), 127.7 (C), 125.9 (CH), 116.2 (CH), 108.5 (CH), 101.9 (CH), 60.9 (CH₂), 55.8 (OCH₃), 21.4 (2 CH₃), 14.4 (CH₃); FT-IR (neat) \tilde{v}ₘₐₓ: 3470, 3306, 2982, 2938, 1694, 1599, 1536, 1464, 1425, 1373, 1321, 1233, 1203, 1161, 1024, 847, 773, 748, 710, 664 cm⁻¹; GC-MS (EI) m/z (% relative intensity): M⁺323 (100), 324 (22), 278 (35), 234 (23), 190 (4); Anal. Calcd for C₂₀H₂₁NO₃: C, 74.28; H, 6.55; N, 4.33. Found: C, 74.00; H, 6.78; N, 4.29.

One-pot borylation/Suzuki coupling of 2-methylindole.

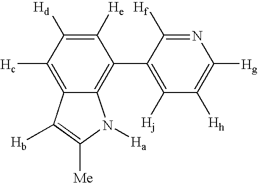

The general borylation procedure was applied to 2-methylindole (131 mg, 1.00 mmol, 1 equiv) and HBPin (218 μL, 192 mg, 1.50 mmol, 1.50 equiv) at 60° C. for 10 h. The GC-FID showed 97% consumption of the starting indole. The reaction mixture was pumped down under high vacuum for 1 h to remove the volatile materials. The Schlenk flask was brought into the glove box, where Pd(PPh₃)₄ (23 mg, 2 mol %), 3-bromopyridine (116 μL, 190 mg, 1.20 equiv), and DME (3 mL) were added. The Schlenk flask was then brought out of the glove box and attached to a Schlenk line. K₃PO₄.nH₂O (319 mg, 1.50 equiv) was added under N₂ counter flow to the Schlenk flask. The flask was stoppered and the mixture was heated at 80° C. for 4 h. The flask was cooled down to room temperature and 5 mL of water were added to the reaction mixture. The reaction mixture was extracted with ether (10 mL×3). The combined ether extractions were washed with brine (10 mL), followed by water (10 mL), dried over MgSO₄ before being concentrated under reduced pressure on a rotary evaporator. Column chromatography (CH₂Cl₂ containing 2% triethylamine) furnished the desired product as a light yellow solid (170 mg, 82% yield, R_{f}=0.3, mp 158-160° C.). ¹H NMR (CDCl₃, 500 MHz): δ 9.10 (br s, 1H, Hₐ), 8.94 (dd, J=2.2, 0.7 Hz, 1H, H_{f}), 8.54 (dd, J=4.9, 1.0 Hz, 1H, H_{g}), 7.88 (ddd, J=7.8, 2.3, 1.7 Hz, 1H, Hᵢ), 7.54 (dt, J=7.8, 0.8 Hz, 1H, H_{c}), 7.40 (ddd, J=7.8, 4.9, 0.8 Hz, 1H, H_{g}), 7.16 (dd, J=7.8, 7.3 Hz, 1H, Hid), 7.08 (dd, J=7.3, 1.0 Hz, 1H, Hₑ), 6.29 (m, 1H, H_{b}), 2.43 (s, 3H, CH₃); ¹³C NMR {¹H} (CDCl₃, 125 MHz): δ 149.2 (CH), 148.3 (CH), 136.0 (C), 135.7 (CH), 135.4 (C), 133.9 (C), 129.7 (C), 124.0 (CH), 121.2 (CH), 121.1 (C), 120.2 (CH), 119.8 (CH), 101.0 (CH), 13.7 (CH₃); FT-IR (neat) \tilde{v}ₘₐₓ: 3200, 2916, 2850, 1574, 1539, 1412, 1280, 1140, 1028, 797, 738, 712 cm⁻¹; GC-MS (EI) m/z (% relative intensity): M⁺208 (100), 193 (3), 180 (9), 166 (2), 152 (4); Anal. Calcd for C₁₄H₁₂N₂: C, 80.74; H, 5.81; N, 13.45. Found: C, 80.10; H, 5.98; N, 13.27. HRMS (FAB): m/z 209.1080 [(M+1⁺); Calcd for C₁₄H₁₃N₂: 209.10788].

### Diborylation of N-methylindole

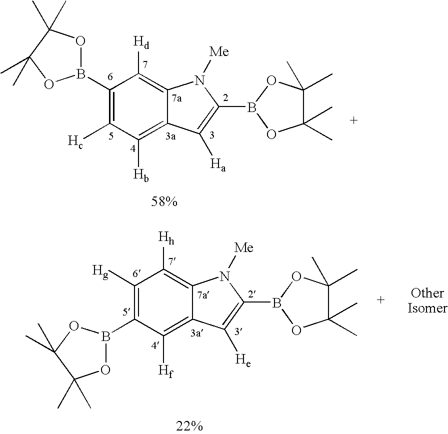

The general procedure was applied to N-methylindole (256 μL, 262 mg, 2.00 mmol, 1 equiv) and HBPin (870 μL, 768 mg, 6.00 mmol, 3.00 equiv) at 60° C. for 24 h. Three diborylated products were observed by GC-FID, their GC retention times and percentage of the isomer mixture are given in the following table.

| | Isomer | | |
|---|---|---|---|
| | A | B | C |
| GC Ret. Time | 19.50 | 21.60 | 22.50 |
| (Min) | | | |
| GC Percentage | 20 | 58 | 22 |

Volatile materials were removed on a rotary evaporator. Gradient column chromatography (hexanes:CH₂Cl₂ 50:50→CH₂Cl₂) was used to obtain two fractions which were significantly enriched in isomer B and C respectively. These two fractions were sufficiently enriched in isomer B and C respectively, to identify and assign their regiochemistry by NMR.

Isomer B: (GC retention time 21.6 min) ¹H, ¹³C NMR, gHMQC and gHMBC spectroscopy showed this major diborylated isomer B to be 2,6-bis(4,4,5,5-tetramethyl-1,3,2-dioxaboryl)-N-methylindole. ¹H NMR (C₆D₆, 500 MHz): δ 8.27 (s, 1H, H_{d}), 8.08 (d, J=7.9 Hz, 1H, Hₐ), 7.70 (dd, J=7.9, 0.9 Hz, 1H, H_{b}), 7.51 (d, J=0.9 Hz, 1H, Hₐ), 3.66 (s, 3H, CH₃), 1.19 (s, 12H, CH₃ of BPin), 1.06 (s, 12H, CH₃ of BPin), ¹H NMR (CDCl₃, 500 MHz): δ 7.84 (d, J=0.7 Hz, 1H, H_{d}), 7.62 (dd, J=8.0, 1.0 Hz, 1H, H_{b/c}), 7.49 (dd, J=8.0, 1.0 Hz, 1H, H_{b/c}), 7.09 (d, J=0.7 Hz, 1H, Hₐ), 3.99 (s, 3H, CH₃), 1.36 (s, 12H, CH₃ of BPin), 1.35 (s, 12H, CH₃ of BPin); ¹³C NMR {¹H} (C₆D₆, 125 MHz): δ 140.6 (C, C7a), 131 (C, C3a), 125.9 (CH, C5), 121.5 (CH, C4), 117.8 (CH, C7), 115.3 (CH, C3), 83.6 (2C), 83.5 (2C), 25.1 (4 CH₃ of BPin), 24.8 (4 CH₃ of BPin).

Isomer C: (GC retention time 22.5 min) ¹H spectroscopy showed this minor diborylated isomer C to be 2,5-bis(4,4,5,5-tetramethyl-1,3,2-dioxaboryl)-N-methylindole. ¹H NMR (CDCl₃, 500 MHz): δ 8.14 (s, 1H, H_{f}), 7.67 (dd, J=8.3, 1.0 Hz, 1H, H_{g/h}), 7.30 (d, j=8.3 Hz, 1H, H_{g/h}), 7.12 (d, J=1.0 Hz, 1H, Hₑ), 3.94 (s, 3H, CH₃), 1.34 (s, 24H, CH₃ of BPin).

Fraction significantly enriched in isomer A was not obtained. Pure samples of isomer B and C were not obtained.

In conclusion, Ir-catalyzed borylation provides the first general approach to functionalizing unprotected indoles at C7. Efforts toward further validating the mechanism, expanding the substrate scope, and elaborating the resulting boronate esters are ongoing.

### REFERENCES CITED

(1) (a) Sundberg, R. J. In Comprehensive Heterocyclic Chemistry; Katritzky, A. R., Rees, C. W., Eds. Pergamon Press: Oxford, 1984; Vol. 4, pp 314-476; (b) Jones, R. A. In Comprehensive Heterocyclic Chemistry; Katritzky, A. R., Rees, C. W., Eds. Pergamon Press: Oxford, 1984; Vol. 4, pp 201-312.
(2) (a) Deprez, N. R.; Kalyani, D.; Krause, A.; Sanford, M. S. J. Am. Chem. Soc. 2006, 128, 4972-4973; (b) Lane, B. S.; Brown, M. A.; Sames, D. J. Am. Chem. Soc. 2005, 127, 8050-8057; (c) Wang, X.; Lane, B. S.; Sames, D. J. Am. Chem. Soc. 2005, 127, 4996-4997; (d) Sezen, B.; Sames, D. J. Am. Chem. Soc. 2003, 125, 5274-5275; (e) Ishiyama, T.; Nobuta, Y.; Hartwig, J. F.; Miyaura, N. Chem. Commun. 2003, 2924-2925; (f) Takagi, J.; Sato, K.; Hartwig, J. F.; Ishiyama, T.; Miyaura, N. Tetrahedron Lett. 2002, 43, 5649-5651; (g) Ishiyama, T.; Takagi, J.; Hartwig, J. F.; Miyaura, N. Angew. Chem. Int. Ed. 2002, 41, 3056-3058.
(3) (a) Govek, S. P.; Overman, L. E. J. Am. Chem. Soc. 2001, 123, 9468-9469; (b) Deng, H. B.; Jung, J. K.; Liu, T.; Kuntz, K. W.; Snapper, M. L.; Hoveyda, A. H. J. Am. Chem. Soc. 2003, 125, 9032-9034; (c) Nicolaou, K. C.; Chen, D. Y. K.; Huang, X. H.; Ling, T. T.; Bella, M.; Snyder, S. A. J. Am. Chem. Soc. 2004, 126, 12888-12896; (d) Garg, N. K.; Sarpong, R.; Stoltz, B. M. J. Am. Chem. Soc. 2002, 124, 13179-13184; (e) Lin, S.; Yang, Z.-Q.; Kwok, B. H. B.; Koldobskiy, M.; Crews, C. M.; Danishefsky, S. J. J. Am. Chem. Soc. 2004, 126, 6347-6355.
(4) Enzymatic chlorinations at C7 in indole^{4a} and tryptophan^{4b} are known: (a) Wiesner, W.; Vanpee, K. H.; Lingens, F. FEBS Lett. 1986, 209, 321-324; (b) Dong, C. J.; Flecks, S.; Unversucht, S.; Haupt, C.; van Pee, K. H.; Naismith, J. H. Science 2005, 309, 2216-2219.
(5) Low yielding Rh-catalyzed carbene insertion into the C7 C—H of a 3-substituted indole has been claimed: (a) Kennedy, A. R.; Taday, M. H.; Rainier, J. D. Org. Lett. 2001, 3, 2407-2409; Aluminum anilide has been reported to mediate ethylene insertion into the C7 C—H bond of 2-methylindole at 280-300° C.: (b) Stroh, R., Hahn, W. Justus Liebigs Ann. Chem. 1959, 623, 176-183.
(6) Hartung, C. G.; Fecher, A.,; Chapell, B.; Snieckus, V. Org. Lett. 2003, 5, 1899-1902.
(7) (a) Tse, M. K.; Cho, J.-Y.; Smith, M. R., III Org. Lett. 2001, 3, 2831-2833; (b) Cho, J. Y.; Tse, M. K.; Holmes, D.; Maleczka, R. E., Jr.; Smith, M. R., III Science 2002, 295, 305-308.
(8) (a) Ishiyama, T.; Takagi, J.; Ishida, K.; Miyaura, N.; Anastasi, N. R.; Hartwig, J. F. J. Am. Chem. Soc. 2002, 124, 390-391; (b) Boller, T. M.; Murphy, J. M.; Hapke, M.; Ishiyama, T.; Miyaura, N.; Hartwig, J. F. J. Am. Chem. Soc. 2005, 127, 14263-14278.
(9) An intriguing alternative to this latter mechanism, involving coordination of the indole N to B in one of the boryl ligands, is not shown.
(10) Chotana, G. A.; Rak, M. A.; Smith, M. R., III J. Am. Chem. Soc. 2005, 127, 10539-10544.
(11) A decrease in selectivity for benzofuran vs. indole is consistent with replacing the indole NH with a less basic O in a heteroatom chelation mechanism.
(12) Even though C3 is the thermodynamic site of protonation in indole,^{12a} the kinetic accessibility of the N lone pair is reflected by the fact that acid catalyzed deuterium exchange at N is ca. 100 times faster than at C3.^{12b} For both sites, experimental evidence strongly supports exchange via an S_{E}2 mechanism^{12b}: (a) Hinman, R. L.; Whipple, E. B. J. Am. Chem. Soc. 1962, 84, 2534-2539; (b) Muir, D. M.; Whiting, M. C. J. Chem. Soc.-Perkin Trans. 2 1976, 388-392.
(13) Prieto, M.; Zurita, E.; Rosa, E.; Munoz, L.; Lloyd-Williams, P.; Giralt, E. J. Org. Chem. 2004, 69, 6812-6820.
(14) Uson, R.; Oro, L. A.; Cabeza, J. A. Inorg. Synth. 1985, 23, 126-130.
(15) (a) Nazare, M.; Schneider, C.; Lindenschmidt A.; Will, D. W. Angew. Chem. Int. Ed. 2004, 43, 4526-4528. (b) Hartung C. G.; Fecher, A.; Chapell, B.; Snieckus, V. Org. Lett. 2003, 5, 1899-1902.
(16) Boller, T. M.; Murphy, J. M.; Hapke, M.; Ishiyama, T.; Miyaura, N.; Hartwig, J. F. J. Am. Chem. Soc. 2005, 127, 14263-14278.
(17) Ma, C.; Liu, X.; Li, X.; Anderson, J. F.; Yu, S.; Cook, J. M. J. Org. Chem. 2001, 66, 4525-4542.
(18) Ishiyama, T.; Nobuta, Y.; Hartwig, J. F.; Miyaura, N. Chem. Commun. 2003, 2924-2925.

The indoles of the present invention are intermediates to natural cytotoxic compounds which have anticancer and antiviral activity. The compounds are also intermediates to synthetic anticancer and antiviral agents based upon the 2-substituted-7-boryl indoles as an intermediate.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

We claim:

1. A process for producing a 2-substituted-7-boryl indole (I), which comprises:
(a) reacting an indole (II) with an unprotected ring nitrogen in a reaction mixture with a non-reactive solvent, selected from, but not limited to, aliphatic hydrocarbons and ethers at temperatures between about 0 and 150° C. with an HB or B-B organic compound, in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium, BY is a boron moiety and the ligand is selected from the group consisting of a phosphorus organic ligand, an organic amine, an imine, a nitrogen heterocycle, and an ether wherein the ligand is at least in part bonded to the iridium, to form the 2-substituted-7-boryl indole (I) in the reaction mixture; and
(b) evaporating the solvent and portions of the reaction mixture which are volatile from the reaction mixture to produce the 2-substituted-7-boryl indole (I).

2. A process for producing a 2-substituted-7-boryl indole (I), which comprises:
(a) reacting an indole (II) with an unprotected ring nitrogen with an HB or B-B organic compound in a reaction mixture with a non-reactive first solvent selected from, but not limited to, aliphatic hydrocarbons and ethers at elevated temperatures between about 0 and 150° C. in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium, BY is a boron moiety and the ligand is selected from the group consisting of a phosphorus organic ligand, an organic amine, an imine, a nitrogen-heterocycle, and an ether wherein the ligand is at least in part bonded to the iridium, in a molar ratio of complex to ligand between 1 to 3 and 1 to 1, wherein the ligand is at least in part bonded to the iridium, to form the 2-substituted-7-boryl indole (I);
(b) evaporating the first solvent and portions of the reaction mixture which are volatile from the reaction mixture;
(c) dissolving the 2-substituted-7-boryl indole in a second solvent; and
(d) isolating the 2-substituted-7-boryl indole (I) from the second solvent.

3. A 2-substituted-7-boryl indole (I) with an unprotected ring nitrogen, wherein there is at least one ring substituent in the 2 position other than hydrogen selected from the group consisting of boryl, halo, cyano, alkyl, alkoxy, thioalkyl, amino alkyl, acyl, alkyl aminoacyl, trifluoro alkyl, aryl, alkyl silyl, and containing 1 to 8 carbon atoms except for the halo group and boryl group and the boryl group is derived from HBPin or B₂Pin.

4. The compound of claim 3 wherein the indole (II) is of the formula: 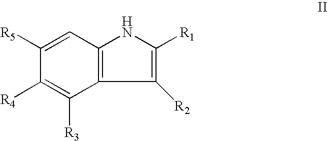
wherein the 2-substituent is R₁ and wherein R₂, R₃, R₄ and R₅ are each selected from the group consisting of hydrogen and the ring substituents for R₁ and wherein the 2-substituted-7-boryl indole (I) is of the formula:

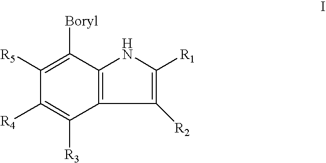

5. A process for producing 2-substituted-7-boryl indole (I), which comprises:
(a) reacting an indole (II) with an unprotected ring nitrogen with HBPin or B₂Pin₂, in a reaction mixture with a non-reactive first solvent selected from, but not limited to, aliphatic hydrocarbons and ethers at elevated temperatures between about 0 and 150° C. in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium BY is a boron moiety and the ligand is selected from the group consisting of a phosphorus organic ligand, an organic amine, an imine, a nitrogen heterocycle, and an ether wherein the ligand is at least in part bonded to the iridium, in a molar ratio of complex to ligand between 1 to 3 and 1 to 1, and wherein the ligand is at least in part bonded to the iridium, to form the 2-substituted-7-boryl indole (I) in the reaction mixture; and
(b) evaporating the solvent and portions of the reaction mixture which are volatile from the reaction mixture to produce the 2-substituted-7-boryl indole (I).

6. The process of claim 5 wherein there is at least one ring substituent for 2-substituted other than hydrogen selected from the group consisting of boryl, halo other than fluoro, cyano, alkyl, alkoxy, thioalkyl, amino alkyl, acyl, alkyl aminoacyl, trifluoro alkyl, aryl, alkyl silyl, and containing 1 to 8 carbon atoms except for the halo group and boryl group and the boryl group is derived from HBPin or B₂Pin.

7. The process of claim 5 or 6 wherein the ligand is selected from the group consisting of: 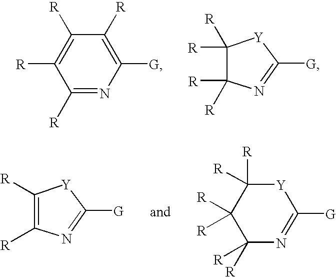
wherein R are each selected from the group consisting of hydrogen, linear alkyl containing 1 to 8 carbon atoms, branched alkyl containing 1 to 8 carbons, and a carbon in a cyclic structure, Y is a carbon, oxygen, nitrogen, or sulfur containing moiety, and G is a heteroatom containing group, multiple atom chain, or multiple atom ring.

8. The process of claim 5 or 6 wherein the ligand is selected from the group consisting of: 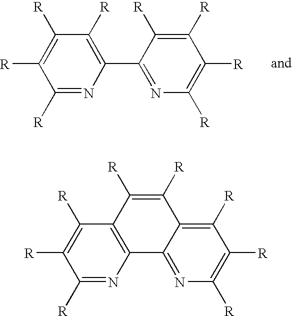
wherein R are each selected from the group consisting of hydrogen, linear alkyl containing 1 to 8 carbon atoms, branched alkyl containing 1 to 8 carbons, and a carbon in a cyclic structure.

9. The process of claim 5 or 6 wherein the ligand is selected from the group consisting of: 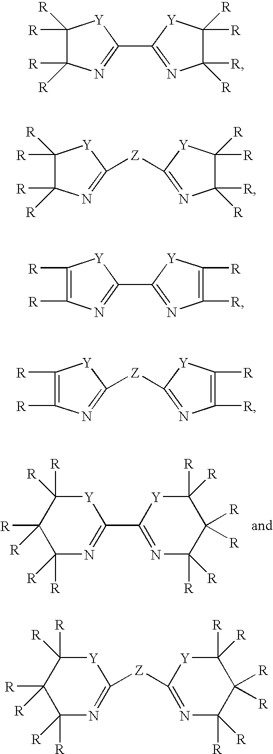
wherein R are each selected from the group consisting of hydrogen, linear alkyl containing 1 to 8 carbon atoms, branched alkyl containing 1 to 8 carbons, and a carbon in a cyclic structure, Y is a carbon, oxygen, nitrogen, or sulfur containing moiety, and Z is a carbon, oxygen, nitrogen, sulfur, or boron containing moiety or a multiple atom chain containing a carbon, oxygen, nitrogen, sulfur, or boron containing moiety.

10. The process of claim 5 or 6 wherein the ligand is selected from the group consisting of: 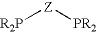
wherein R are each selected from the group consisting of hydrogen, aryl, linear alkyl containing 1 to 8 carbon atoms, branched alkyl containing 1 to 8 carbons, alkoxy, or a carbon in a cyclic structure and Z is a carbon, oxygen, or nitrogen containing moiety or a multiple atom chain containing a carbon, oxygen, or nitrogen containing moiety.

11. The process of claim 1 or 2 wherein the HB or B-B organic compound is HBPin or B₂Pin₂.

12. The process of claims 1 or 2 wherein the complex is an iridium complex of [Ir(OMe)(COD)]₂, [Ir(Cl)(COD)]₂, or (COD) (η⁵-indenyl)Ir, where COD is 1,5-cyclooctadine, complexed with 4,4-di-t-butyl-2,2′bipyridine (dtbpy).

13. The process of claims 1 or 2 wherein the complex is an iridium complex of [Ir(OMe)(COD)]₂, [Ir(Cl)(COD)]₂, or (COD) (η⁵-indenyl)Ir, where COD is 1,5-cyclooctadine, complexed with 1,2-bis(dimethylphospino)ethane.

14. The process of claim 1 wherein when indole (I) is reacted with an aryl halide to form a 2-substituted-7-aryl indole.
